(19) United States
(12) Patent Application Publication
(10) Pub. No.: US 2025/0073421 A1
(43) Pub. Date: Mar. 6, 2025

(54) TWO-STAGE SEPARABLE SUCTION CATHETER SYSTEM

(71) Applicant: Clayton S. Garthe, Bend, OR (US)

(72) Inventor: Clayton S. Garthe, Bend, OR (US)

(21) Appl. No.: 18/820,918

(22) Filed: Aug. 30, 2024

Related U.S. Application Data
(60) Provisional application No. 63/536,203, filed on Sep. 1, 2023.

(51) Int. Cl. A61M 25/00; A61M 1/00

(52) U.S. Cl. CPC A61M 25/0026; A61M 1/71

(57) ABSTRACT

A two-stage suction catheter for rapidly clearing liquids and solids from the upper airway has a first catheter stage configured to regulate effective moderate level negative pressure level at the distal end inlet and a high-capacity catheter stage having a large aperture size that is configured to regulate effective high level negative pressure level at the high-capacity catheter distal connection inlet when the first catheter stage is separated from the high-capacity catheter stage. The two-stage suction catheter has an attachment arrangement comprising a detent that releasably the first catheter stage to the high-capacity catheter stage, wherein the detent is configured to permit separation of the stages by a one-handed separation action. Methods and systems are also described. A suction cannister lid has a high capacity port and auxiliary port that may be separately sealed by a removable cap or insertion of high-capacity tubing, respectively.

### RELATED APPLICATION

The present nonprovisional patent application claims the benefit of commonly owned provisional Application having Ser. No. 63/536,203, filed on Sep. 1, 2023, which provisional Application is incorporated herein by reference in its entirety.

### FIELD

The present invention relates a medical suction system that is designed to rapidly clear liquids and solids from the upper airway.

### BACKGROUND

In emergency resuscitation, protecting a person's airway is a critical first step to save the life of a patient. To do this, an endotracheal tube must be placed past the vocal cords. Often the airway above the vocal cords is blocked by obstructions including liquids (mucus, phlegm, blood, stomach contents), solids (food, broken teeth, foreign bodies) or both. Medical suction systems are used to rapidly clear these obstructions.

Health care facilities, such as hospitals, emergency rooms and urgent care facilities, are provided with vacuum supply for use with medical suction systems, which is provided by a centrally located vacuum generator (or a pump) or by a free-standing device. Such vacuum supply may have a vacuum pressure of, for example, from about 150 to about 450 mmHg, or from about 200 to about 450 mmHg.

### SUMMARY

It has been found that conventional suction systems that are ubiquitous in nearly all resuscitation environments are designed to clear low viscosity liquids but are ineffective in emergency conditions when solids or viscous liquids are encountered when attempting to clear the airway of a patient. Conventional medical suction systems to be connected to vacuum supply are purposefully configured with design features including a suction device tip with a small diameter suction aperture (e.g., having an aperture size inner diameter of from about 5 to about 7 mm) and pressure control features to provide an effective vacuum pressure at the suction device tip of about 80 mmHg. These design parameters are generally adequate for use in most emergency resuscitation situations, and in fact is appropriate to avoid damage to patient tissues that could be caused by application of excessive suction.

However, even though the need for high-capacity suction that can clear solids or viscous liquid is infrequent, when such a scenario arises the consequences of not having high suction immediately available are dire-literally life or death. Hundreds to thousands of deaths each year could be avoided by making rapidly accessible high-capacity suction available in emergency care facilities. High capacity, high vacuum pressure medical suction systems are available, and must be used carefully to avoid tissue damage to the patient during use.

Medical practitioners almost never know if they will need high-capacity suction until the moment of intubation, when materials such as liquids, solids or both as discussed above are suddenly presented. In such a situation, it is critically important to be able to switch rapidly from standard suction to high-capacity suction. The ability to quickly transition to a different suction arrangement in the medical treatment setting is further complicated by the fact that the practitioner is fully engaged in a stressful moment of patient care with the suction device in one hand and often another instruments, such as a laryngoscope, in the other hand. Providing two separate suction systems of different capacities in the treatment setting is not a solution, because changing to a different suction system (even if it were present and reliably operational), is clumsy and slow.

As a practical matter, a medical practitioner only gets one chance to secure an airway. It cannot be emphasized enough that seconds matter in the urgent moment when the airway is unexpectedly blocked by solids or viscous liquid.

Vandenberg U.S. Pat. No. 5,921,970 describes a large variable diameter medical suction system comprising a nozzle secured to a handle by a “quick release” attachment means including “the twist lock pin and channel combination shown, friction joined fittings, matting screw threads, and latches.” See column 6, lines 3-11. The demonstrated attachment means of Vandenberg requires a two-handed twisting operation to separate the nozzle from the handle, which is simply too slow and cumbersome for the practitioner to carry out effectively in real life emergency situations.

The present two-stage suction catheter enables a practitioner applying suction at conventional suction vacuum levels to quickly convert to application of high-capacity suction with one hand, so that at the same time the practitioner's other hand is free for manipulating a laryngoscope (or other device being used at the time of the procedure) without assistance and without having to look away from the airway. Advantageously, the present two-stage suction catheter enables a practitioner to quickly convert to application of high-capacity suction without assistance of another person. In the moment of the medical crisis, another person may not be physically close enough to provide immediate assistance, or precious time may be lost while attempting to communicate to a second person exactly what assistance is required for switching from one suction system to another. The present two-stage suction catheter thus provides practitioners the ability to apply necessary high-capacity suction with immediacy that was not previously attainable.

In an embodiment, a two-stage suction catheter is provided for rapidly clearing liquids and solids from the upper airway. The two-stage suction catheter comprises a first catheter stage and a high-capacity catheter stage. The first catheter stage has a tapered lumen extending from a distal end inlet to an attachment end outlet and a longitudinal axis defined as a line passing through the center of the lumen. The distal end inlet has an aperture size inner diameter of from about 5 mm to about 7 mm and the attachment end outlet has an aperture size inner diameter of from about 14 mm to about 18 mm.

The first catheter stage is provided with an airflow vent configured to regulate effective negative pressure level at the distal end inlet when the two-stage suction catheter is connected to a vacuum supply.

In an embodiment, when the two-stage suction catheter is fully assembled to use the first catheter stage as the patient contact suction tip, the first catheter stage comprises a first catheter stage airflow vent configured to provide an effective negative pressure level of from about 50 to about 100 mmHg at the distal end inlet when the two-stage suction catheter is connected to a standard medical care facility vacuum supply. This low negative pressure, in combination with a distal end inlet aperture size inner diameter of from about 5 to about 7 mm, provides effective suction for most ordinary procedures.

The high-capacity catheter stage has a lumen extending from a distal connection inlet to a proximal end outlet, wherein the distal connection inlet has an aperture size inner dimension matching the aperture size inner dimension of the first catheter stage attachment end outlet, and the proximal end outlet has an aperture size inner dimension of from about 14 to about 18 mm.

It has been found that the high-capacity catheter stage inner dimensional size of from about 14 to about 18 mm is effective for handling the solids and liquid that must be cleared in emergency situations as discussed above. In particular, it has been found that the narrowest portion of the esophagus is most adults is 15-16 mm in diameter, and so a high-capacity catheter stage having this inner dimensional size will accommodate most if not all regurgitated food and vomitus. This inner dimensional sized also is larger than all teeth (including roots) except the incisors. However, teeth encountered in these settings are almost always broken, and given the oblong shape of an incisor, even if it is intact it is expected to be easily cleared away, oriented in line with the flow of suction.

The high-capacity catheter stage comprises a high-capacity catheter stage ventilation aperture, wherein the high-capacity catheter stage ventilation aperture is configured to regulate effective negative pressure level at the high-capacity catheter distal connection inlet when the first catheter stage is separated from the high-capacity catheter stage and the high-capacity catheter stage is connected to a vacuum supply. Because of the larger aperture sizes associated with the high-capacity catheter stage, the two-stage suction catheter when employed after removal of the first catheter stage provides both higher negative pressure (e.g., a level of from about 100 to about 200 mmHg at the distal connection inlet when connected to a standard medical care facility vacuum supply) and effective volume flow capacity so that pieces do not clog the catheter.

An attachment arrangement comprises a detent that releasably connects the attachment end outlet of the first catheter stage to the distal connection inlet of the high-capacity catheter stage, wherein the detent is configured to permit separation of the first catheter stage from the high-capacity catheter stage by a one-handed separation action.

For purposes of the present discussion, a detent is a mechanical or magnetic component configured to resist separation of the first catheter stage from the high-capacity catheter stage, wherein the detent may be overcome to permit separation of the first catheter stage from the high-capacity catheter stage. In an embodiment, the detent is a friction fitting of the attachment end outlet of the first catheter stage to the distal connection inlet of the high-capacity catheter stage that is configured to be overcome by imparting a force to the first catheter stage. In an embodiment, the detent comprises one or more physical projections, such as knobs or tabs on one catheter stage which interface with corresponding notches on the other catheter stage to releasably connect the catheter stages together. In an embodiment, the detent comprises one or more physical projections, such as knobs or tabs on one catheter stage which resist movement of a sliding attachment arrangement that can be overcome by application of sufficient force to permit transit of the sliding attachment arrangement to permit separation of the first catheter stage from the high-capacity catheter stage. Various embodiments of the attachment arrangement are described herein.

For purposes of the present discussion, a one-handed separation action is an action carried out by a practitioner that achieves separation of the first catheter stage from the high-capacity catheter stage without using the practitioner's other hand to grip a portion of the two-stage suction catheter or attached equipment to impart the separation force, and without the assistance of a hand of a second person to grip a portion of the two-stage suction catheter or attached equipment to impart the separation force.

In an embodiment, separation of the first catheter stage from the high-capacity catheter stage may be carried out by imparting a force to the first catheter stage that overcomes the detent, wherein the force has a lateral component relative to the longitudinal axis of the first catheter stage.

In an embodiment, separation of the first catheter stage from the high-capacity catheter stage may be carried out by imparting a force to the first catheter stage that overcomes the detent, wherein the force has a longitudinal component parallel to the longitudinal axis of the first catheter stage in the direction away from the high-capacity catheter stage.

In an embodiment the attachment arrangement comprises a release switch configured to disable operation of the detent and permit separation of the first catheter stage from the high-capacity catheter stage. Embodiments that comprise a release switch provide an additional advantage of secure connection of the catheter stages with reduced likelihood of inadvertent separation by accidental bumping of the two-stage suction catheter on a surface.

In an embodiment, separation of the first catheter stage from the high-capacity catheter stage may be achieved during or after actuating the release switch by application of a force to the first catheter stage in a manner as described above.

In an embodiment, the attachment arrangement comprises a biasing member configured to apply a force to urge separation of the first catheter stage from the high-capacity catheter stage upon actuation of the release switch.

In an embodiment, the present two-stage suction catheter has no moving pieces.

In an embodiment, the high-capacity tubing to be used with the high-capacity catheter stage as discussed herein is a low-friction tubing having the same inner dimensional size of from about 14 to about 18 mm.

A method of rapidly clearing liquids and solids from the upper airway is also provided that comprises applying suction to a patient in need thereof with the two-stage suction catheter as described herein using the first catheter stage. Upon recognition that enhanced suction is required to rapidly clear liquids and solids from the upper airway, the first catheter stage is detached from the high-capacity catheter stage in a one-handed separation action, and suction is applied to a patient in need thereof with the high-capacity catheter stage of the two-stage suction catheter.

Optionally, after the airway is cleared and secured, it is possible to return to a standard suction configuration by reassembling the original first catheter stage with the high-capacity catheter stage, or alternatively assembling a replacement first catheter stage with the high-capacity catheter stage. In an embodiment the first catheter stage and the high-capacity catheter stage are provided with markings and/or physical features to ensure proper orientation of the respective stages for ease of reassembly.

A system for rapidly clearing liquids and solids from the upper airway is also provided that comprises the above described two-stage suction catheter; a suction cannister system comprising a suction cannister and suction cannister lid. The suction cannister lid comprises a high capacity port. High-capacity tubing is configured to connect the proximal end outlet of the high-capacity catheter stage of the two-stage suction catheter to the high capacity port to form an airtight coupling seal from the two-stage suction catheter to the high capacity port.

A suction cannister lid is also provided comprising a wall suction port configured for attachment to a vacuum supply and a vacuum port array. The vacuum port array comprises a body portion formed as a hollow cylinder extending from a top surface of the cannister lid defining a high capacity port providing fluid communication through the cannister lid. An auxiliary port extends laterally from the body portion. The high capacity port is configured to receive high-capacity tubing inserted into the high capacity port to form an airtight coupling seal permitting flow of air and suctioned material through the high capacity port and simultaneously occluding the auxiliary port to prevent air flow through the auxiliary port. A removable cap is provided that is configured to seal the high capacity port while leaving the auxiliary port open permitting flow of air and suctioned material through the auxiliary port.

The present two-stage suction catheter and system provides a number of significant advantages. First, the present highly adaptable system for rapidly clearing liquids and solids from the upper airway is configured to work with conventional vacuum supply systems already provided in existing hospital infrastructure, such as the “wall suction” systems commonly used in emergency rooms and urgent care centers.

Second, the present two-stage suction catheter and system is designed to be as safe and effective as existing products for the majority of resuscitations.

Third, the present two-stage suction catheter and system has capacity to rapidly clear viscous liquids, solids, and complex mixtures, maximizing life-saving potential for all resuscitations.

Fourth, the present two-stage suction catheter is configured to be capable of directing all available negative pressure (conventionally 200 mmHg negative pressure gradient in emergency care facilities) when operating as a high-capacity catheter, while at the same time being controllable to provide a suitable, lower suction force as needed for safe aspiration of fluids and materials from patients when lower suction force is sufficient and desirable to prevent tissue damage that might be experienced at higher suction levels.

Fifth, the present two-stage suction catheter and system provides a highly effective, adaptable suction system that can be provided at low cost. Thus, the present two-stage suction catheter and system can be provided at a cost point that is very attractive and achievable for use in medical care facilities having a very limited budget.

### BRIEF DESCRIPTION OF THE DRAWINGS

The accompanying drawings, which are incorporated in and constitute a part of this application, illustrate several aspects of the invention and together with a description of the embodiments serve to explain the principles of the invention. A brief description of the drawings is as follows:

FIG. 1 is a cross-section view of a two-stage suction catheter as described herein.

FIG. 2a is a side view of the two-stage suction catheter as shown in FIG. 1.

FIG. 2b is a side view of the first catheter stage of the two-stage suction catheter as shown in FIG. 2a after separation of the two stages.

FIG. 2c is a side view of the high-capacity catheter stage of the two-stage suction catheter as shown in FIG. 2a after separation of the two stages.

FIG. 3a is a side view of an alternate design of a two-stage suction catheter as described herein.

FIG. 3b is a top view of the first catheter stage of the two-stage suction catheter as Shown in FIG. 3a after separation of the two stages.

FIG. 3c is a side view of the first catheter stage of the two-stage suction catheter as shown in FIG. 3a after separation of the two stages.

FIG. 3d is a side view of the high-capacity catheter stage of the two-stage suction catheter as shown in FIG. 3a after separation of the two stages.

FIG. 3e is a top sectional view of the first catheter stage and the high-capacity catheter stage of the two-stage suction catheter as shown in FIG. 3a after separation of the two stages, showing the interlocking features of the attachment arrangement.

FIG. 4a is a side view of a two-stage suction catheter as described herein where the attachment arrangement comprises break-away release prior to separation of the two stages.

FIG. 4b is a side view of a two-stage suction catheter as shown in FIG. 4a after separation of the two stages.

FIG. 5a is a side view of a two-stage suction catheter as described herein where the attachment arrangement wherein a portion of the attachment end of the first catheter stage is cut away to receive the distal portion of the high-capacity catheter stage in a nested arrangement.

FIG. 5b is a side view of the two-stage suction catheter as shown in FIG. 5a after separation of the two stages.

FIG. 6a is a side view of a two-stage suction catheter as described herein where the attachment arrangement comprises a hinged connection.

FIG. 6b is a side view of the two-stage suction catheter as shown in FIG. 6a after separation of the two stages.

FIG. 7a is a side view of a two-stage suction catheter as described herein where the attachment arrangement comprises a hinged connection in an alternate hinge location.

FIG. 7b is a side view of the two-stage suction catheter as shown in FIG. 7a after separation of the two stages.

FIG. 8a is a side view of a two-stage suction catheter as described herein where the attachment arrangement comprises a sliding connection and a hook release-assist.

FIG. 8b is a side view of the two-stage suction catheter as shown in FIG. 8a after separation of the two stages.

FIG. 9 is a side partial cross-section view of a two-stage suction catheter as described herein where the attachment arrangement comprises a thumb activated release.

FIG. 10 is a top view of the two-stage suction catheter as shown in FIG. 9 showing the thumb activated release.

FIG. 11 is a side cross-section view of a two-stage suction catheter as described herein where the attachment arrangement comprises an assisted thumb activated release.

FIG. 12 is a side cross-section view of a two-stage suction catheter as described herein where the attachment arrangement comprises a button release.

FIG. 13 is a top view of the two-stage suction catheter as shown in FIG. 9 showing the button release.

FIG. 14a is a side cross-section view of a two-stage suction catheter as described herein where the attachment arrangement comprises a sliding barrel connection.

FIG. 14b is a side cross-section view of the two-stage suction catheter as shown in FIG. 14a after separation of the two stages.

FIG. 15a is a side view of a two-stage suction catheter as described herein where the attachment arrangement comprises a track connection.

FIG. 15b is a side view of the two-stage suction catheter as shown in FIG. 15a after initial separation of the two stages.

FIG. 15c is a side view of the two-stage suction catheter as shown in FIG. 15a after separation and displacement of the two stages.

FIG. 16 is a perspective view of a canister lid and cannister as described herein.

FIG. 17a is a perspective view of tubing arrangement as described herein.

FIG. 17b is a perspective view of tubing arrangement engaging with a high capacity port as described herein.

### DETAILED DESCRIPTION

The aspects of the present invention described below are not intended to be exhaustive or to limit the invention to the precise forms disclosed in the following detailed description. Rather a purpose of the aspects chosen and described is by way of illustration or example, so that the appreciation and understanding by others skilled in the art of the general principles and practices of the present invention can be facilitated.

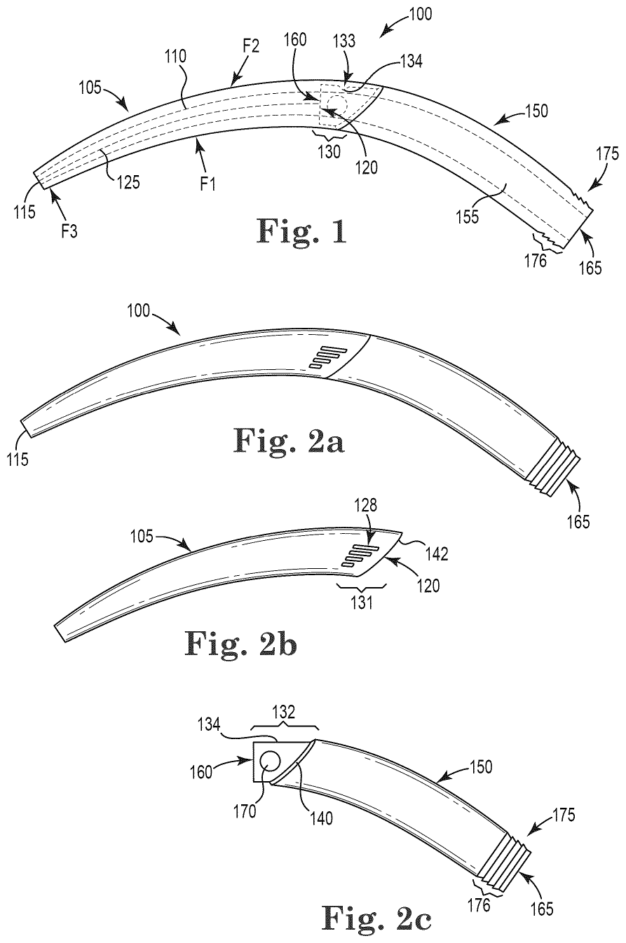

Turning now to the Figures, an embodiment of the present two-stage suction catheter is described with respect to FIGS. 1 and 2a-c, where like reference numerals relate to like parts.

FIG. 1 is a cross-section view of a two-stage suction catheter 100 comprising a first catheter stage 105 and a high-capacity catheter stage 150. As shown, the two-stage suction catheter 100 has a curved shape to accommodate the natural curvature of the oropharyngeal anatomy. In an embodiment, the two-stage suction catheter 100 has an arc measure of from about 30 and about 90 degrees. In an embodiment, the two-stage suction catheter 100 has an arc measure of from about 30 and about 70 degrees. In an embodiment, the two-stage suction catheter 100 has a nominal radius of from about 3 inches to about 12 inches. In an embodiment, the two-stage suction catheter 100 has a nominal radius of from about 4 inches to about 10 inches. In an embodiment, the two-stage suction catheter 100 has a nominal radius of from about 5 inches to about 12 inches. In an embodiment, the radius of the arc is varied, so that the two-stage suction catheter 100 is in the shape of a gentle curve. In an embodiment, the two-stage suction catheter 100 is straight.

The first catheter stage 105 has a lumen 110 extending from a distal end inlet 115 to an attachment end outlet 120. A first catheter stage longitudinal axis 125 is defined as a line passing through the center of lumen 110. In the case where the first catheter stage 105 has a curved shape as shown, first catheter stage longitudinal axis 125 is likewise a curved axis. Distal end inlet 115 has an aperture size inner diameter from about 5 to about 7 mm, and attachment end outlet 120 has an aperture size inner diameter of from about 14 to about 18 mm.

High-capacity catheter stage 150 likewise has a lumen 155 extending from a distal connection inlet 160 to a proximal end outlet 165, wherein the distal connection inlet 160 has an aperture size inner dimension matching the aperture size inner dimension of the first catheter stage attachment end outlet, so that when first catheter stage 105 and a high-capacity catheter stage 150 are connected lumen 110 and lumen 155 join to form a continuous lumen. In an embodiment, the continuous lumen formed by the combined lumen 110 and lumen 155 has a smooth, low-friction inner surface. It has been found that irregular surfaces in the lumen decrease flow and suction force, causing material to collect in the tubing, which further limits flow, which further allows material to collect, which further limits flow, etc., creating a cycle that causes the entire system to fail.

Proximal end outlet 165 has an aperture size inner dimension of from about 14 to about 18 mm. In an aspect, the cross section of lumen 110 and lumen 155 gradually increases from distal end inlet 115 to proximal end outlet 165 to minimize the likelihood that the two-stage catheter will be blocked by obstructive liquids and/or solids. In an embodiment, the combined lumen formed from lumen 110 and lumen 155 is continually tapered with no intermediate constrictions between from distal end inlet 115 to proximal end outlet 165.

High-capacity catheter stage 150 is provided with connection end 175 to connect proximal end outlet to high-capacity tubing (not shown), which in turn will connect directly or indirectly to a medical vacuum supply system. Connection end 175 may have any connection configuration suitable for connecting to high-capacity tubing. In an embodiment, Connection end 175 is provided with hose barbs 176 for connecting to high-capacity tubing.

Two-stage suction catheter 100 is provided with attachment arrangement 130 connecting attachment end outlet 120 of first catheter stage 105 to distal connection inlet 160 of high-capacity catheter stage 150. Attachment arrangement 130 is configured to release by a one-handed separation action that separates the first catheter stage from the high-capacity catheter stage.

As shown, first catheter stage 105 comprises first extension region 131, and high-capacity catheter stage 150 comprises second extension region 132, wherein the respective extension regions are sized to overlap and fit together to form attachment arrangement 130. As shown, first extension region 131 has an enlarged inner diameter that is sized to fit as a sleeve over the smaller outer diameter of second extension region 132. Protuberant tab 133 is provided on the inner surface first extension region 131 and is configured to engage with corresponding recess 134 provided on the outer surface of second extension region 132 as a detent to resist separation of the first catheter stage from the high-capacity catheter stage. First catheter stage 105 may be separated from high-capacity catheter stage 150 by imparting a force that has a lateral component relative to first catheter stage longitudinal axis 125 at first catheter stage 105, for example, as shown as F1, F2 or F3 in FIG. 1. The force may be applied by, for example, pressing the catheter into an available surface, such as an available table, support stand, bed frame or the mattress of the gurney on which the patient is positioned. In an embodiment, the force may be applied by, for example, pressing the catheter into an available surface of the practitioner's body, such as their thigh, arm, and the like. Removal of first catheter stage 105 in this manner immediately transforms the two-stage suction catheter to a high-capacity catheter.

In an embodiment, diagonally oriented rim 140 is provided on high-capacity catheter stage 150 that fits profile curve 142 of the end of first catheter stage 105 to ensure that the two pieces can only go together one way and guides the two pieces together smoothly. Alternative indication markers or mating structures on first catheter stage 105 and high-capacity catheter stage 150 may be used to facilitate proper assembly or reassembly of the two-stage suction catheter as needed for application of suction at conventional levels after the need for high-capacity suction has passed.

First catheter stage 105 is provided with airflow vent 128 configured regulate effective negative pressure level at the distal end inlet when the fully assembled two-stage suction catheter 100 is connected to a medical vacuum supply. Likewise, high-capacity catheter stage 150 is provided with ventilation aperture 170 configured to regulate effective negative pressure level at the high-capacity catheter distal connection inlet when first catheter stage 105 is separated from high-capacity catheter stage 150 and the high-capacity catheter stage is connected to a vacuum supply. In an embodiment, first catheter stage 105 is provided with a pair of airflow vents on opposite sides of first catheter stage 105. In an embodiment, first catheter stage 105 is provided with a plurality of airflow vents. In an embodiment, high-capacity catheter stage 150 is provided with a pair of ventilation apertures on opposite sides of high-capacity catheter stage 150. In an embodiment, high-capacity catheter stage 150 is provided with a plurality of ventilation apertures.

When the two-stage suction catheter is fully assembled, airflow vent 128 aligns with ventilation aperture 170 so that air can pass through airflow vent 128 and ventilation aperture 170 to prevent development of full vacuum at distal end inlet 115. Reduction of suction by airflow vent 128 in an aspect permits a practitioner to break suction during use, thereby reducing the likelihood of tissue damage. During use, the practitioner can partially or fully block the passage of air through airflow vent 128, for example by partially or fully covering airflow vent 128 with a hand, in order to regulate the amount of suction force delivered at the treatment site.

Likewise, when first catheter stage 105 is separated from high-capacity catheter stage 150, and catheter is used in high-capacity catheter mode, the practitioner can partially or fully block the passage of air through ventilation aperture 170, for example by partially or fully covering ventilation aperture 170 with a hand, in order to regulate the amount of suction force delivered at the treatment site. Reduction of suction by ventilation aperture 170 in an aspect permits a practitioner to break suction during use, thereby reducing the likelihood of tissue damage.

Regulation of the flow of air is advantageous to achieve control of the process of removal of obstructive materials, to maximize effective suction when needed, and also to prevent damage to sensitive tissues (e.g., the vocal cords).

It has additionally been found that when a plurality of airflow vents are provided on first catheter stage 105, the vents can provide enhanced flexibility at the attachment end of first catheter stage 105, easing separation and reconnection of first catheter stage 105 and high-capacity catheter stage 150.

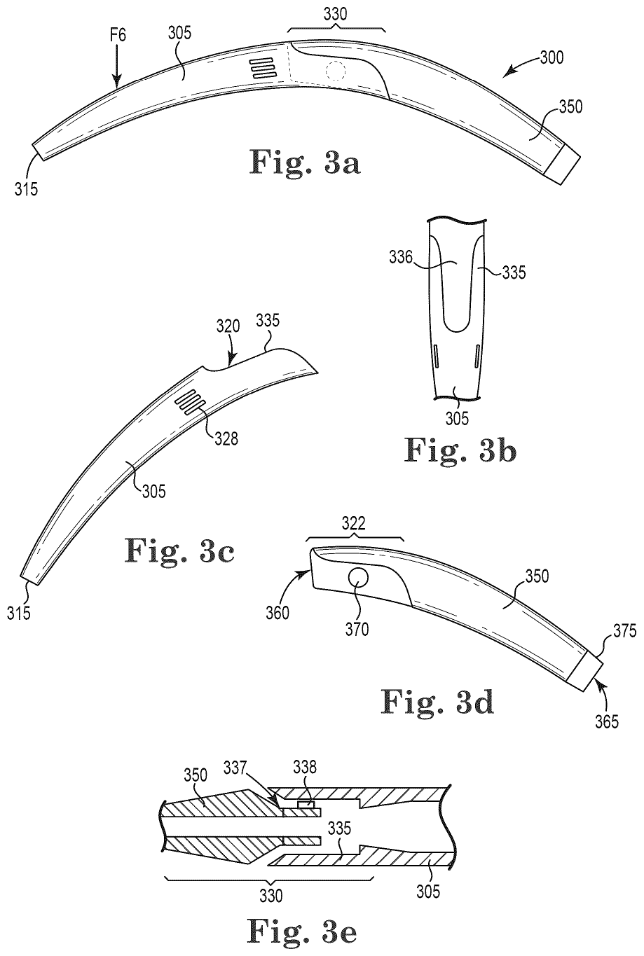

FIG. 3a is a side view of an alternate design of a two-stage suction catheter 300 comprising a first catheter stage 305 and a high-capacity catheter stage 350. As shown, the two-stage suction catheter 300 has a curved shape. In an embodiment, the two-stage suction catheter 300 is straight. FIG. 3c is a side view of the first catheter stage 305 of the two-stage suction catheter 300 as shown in FIG. 3a after separation of the two stages. FIG. 3d is a side view of the high-capacity catheter stage 350 of the two-stage suction catheter 300 as shown in FIG. 3a after separation of the two stages.

First catheter stage 305 has a lumen (not shown) extending from a distal end inlet 315 to an attachment end outlet 320. A first catheter stage longitudinal axis is defined as a line passing through the center of the lumen. In the case where the first catheter stage 305 has a curved shape as shown, first catheter stage longitudinal axis is likewise a curved axis. Distal end inlet 315 has an aperture size inner diameter from about 5 to about 7 mm, and attachment end outlet 320 has an aperture size inner diameter of from about 14 to about 18 mm.

High-capacity catheter stage 350 likewise has a lumen (not shown) extending from a distal connection inlet 360 to a proximal end outlet 365, wherein the distal connection inlet 360 has an aperture size inner dimension matching the aperture size inner dimension of the first catheter stage attachment end outlet, so that when first catheter stage 305 and a high-capacity catheter stage 350 their respective lumens join to form a continuous lumen. In an embodiment, the continuous lumen formed by the combined lumens has a smooth, low-friction inner surface. It has been found that irregular surfaces in the lumen decrease flow and suction force, causing material to collect in the tubing, which further limits flow, which further allows material to collect, which further limits flow, etc., creating a cycle that causes the entire system to fail.

Proximal end outlet 365 has an aperture size inner dimension of from about 14 to about 18 mm. In an aspect, the cross section of the combined lumens gradually increases from distal end inlet 315 to proximal end outlet 365 to minimize the likelihood that the two-stage catheter will be blocked by obstructive liquids and/or solids. In an embodiment, the combined lumen is continually tapered with no intermediate constrictions between from distal end inlet 315 to proximal end outlet 365.

High-capacity catheter stage 350 is provided with connection end 375 to connect proximal end outlet to high-capacity tubing (not shown), which in turn will connect directly or indirectly to a medical vacuum supply system. Connection end 375 may have any connection configuration suitable for connecting to high-capacity tubing.

Two-stage suction catheter 300 is provided with attachment arrangement 330 connecting attachment end outlet 320 of first catheter stage 305 to distal connection inlet 360 of high-capacity catheter stage 350. Attachment arrangement 330 is configured to release by a one-handed separation action that separates the first catheter stage from the high-capacity catheter stage. Aspects of attachment arrangement 330 are described in more detail, in particular by reference to FIG. 3b and FIG. 3c.

FIG. 3b is a top view of first catheter stage 305 of the two-stage suction catheter as shown in FIG. 3a after separation of the two stages. First catheter stage 305 has a connecting partial skirt 335 having a gap 336 to receive the distal portion 322 of the high-capacity catheter stage 350 in a nested arrangement. Partial skirt 335 as shown is an incomplete sleeve extending from the proximal end of first catheter stage 305 to wrap approximately ¾ of the outer circumference of distal portion 322 of high-capacity catheter stage 350. In an embodiment, gap 336 comprises about ¼ of the outer circumference of distal portion 322 of high-capacity catheter stage 350.

FIG. 3e is a top cross-section view of the first catheter stage 305 being connected with high-capacity catheter stage 350.

In an embodiment, a recess 337 is provided in distal portion 322 of high-capacity catheter stage 350 to receive the connecting partial skirt 335 in an interference fit.

The connecting partial skirt 335 may be formed from a flexible material, such as a plastic, that can flex laterally, permitting the gap 336 to widen to at least the size of the outer diameter of the distal high capacity catheter, such that the high capacity catheter may pass through the gap when a laterally directed force (such as shown as F6 in FIG. 3a) is applied to the first stage, permitting one-handed separation of the two stages.

Connecting partial skirt 335 may be advantageously configured to cover and occlude high-capacity catheter stage ventilation aperture 370 when first catheter stage 305 is connected to high-capacity catheter stage 350.

In an embodiment, connecting tabs 338 on the inner surface of connecting partial skirt 335 may be provided that correspond to the ventilation aperture 370 on the high capacity catheter, facilitating a firm connection when assembled. In the embodiment where ventilation apertures 370 are occluded by the connecting partial skirt 335 and/or connecting tabs 338, airflow vents 328 are present on the proximal first stage ahead of the connecting partial skirt 335 for control of vacuum during use of the first catheter stage 305.

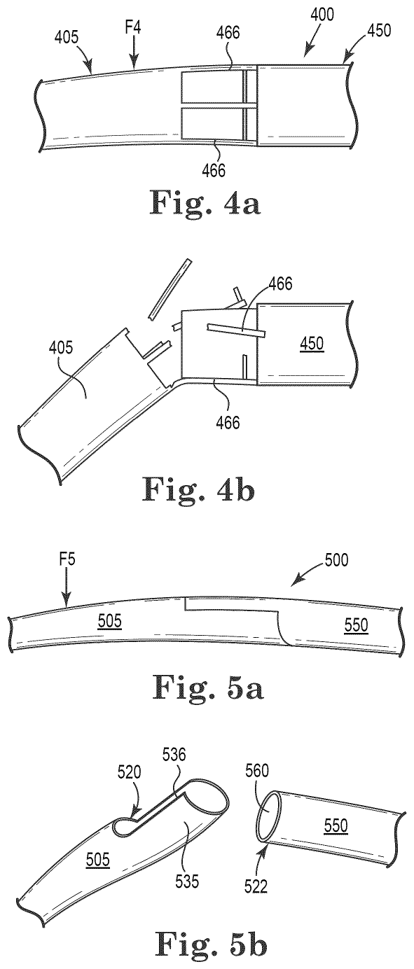

An alternative attachment arrangement is shown in FIG. 4a and FIG. 4b, where the attachment arrangement comprises break-away release prior to separation of the two stages.

FIG. 4a is a side view of a two-stage suction catheter 400 as described herein where the attachment arrangement comprises break-away release prior to separation of the two stages. The respective lumens of first catheter stage 405 and high-capacity catheter stage 450 are coupled, for example by a slip joint or compression joint, and first catheter stage 405 and high-capacity catheter stage 450 are joined by break-away connection struts 436. First catheter stage 405 may be separated from high-capacity catheter stage 450 by imparting a force that has a lateral component relative to first catheter stage longitudinal axis at first catheter stage 405, for example, as shown as F4.

FIG. 4b is a side view of a two-stage suction catheter as shown in FIG. 4a during separation of the two stages. Upon complete separation, high-capacity catheter stage 450 is available for use in applying high capacity suction to rapidly clear liquids and solids as necessary

FIG. 5a is a side view of an alternative attachment arrangement of a two-stage suction catheter 500 wherein the first catheter stage 505 has a connecting partial skirt 535 having a gap 536 to receive the distal cylindrical portion 522 of the high-capacity catheter stage 550 to form a nested partial skirt coupling arrangement, thereby connecting attachment end outlet 520 to distal connection inlet 560. FIG. 5b is a side view of the two-stage suction catheter as shown in FIG. 5a after separation of the two stages by imparting a force that has a lateral component relative to first catheter stage longitudinal axis at first catheter stage 505, for example, as shown as F5 in FIG. 5a.

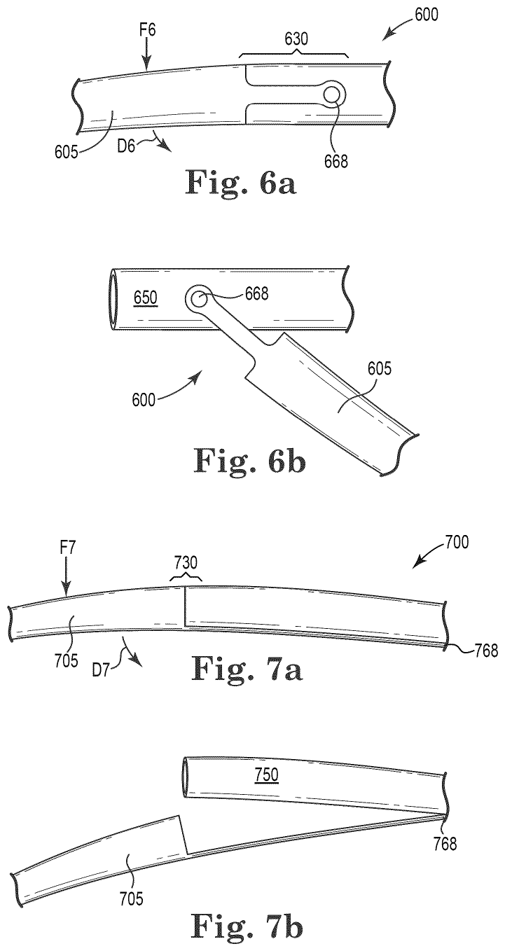

FIG. 6a is a side view of an alternative attachment arrangement of a two-stage suction catheter 600 where the attachment arrangement 630 comprises a hinged connection 668. The respective lumens of first catheter stage 605 and high-capacity catheter stage 650 are coupled by compression and are held together by hinged connection 668. First catheter stage 605 may be separated from high-capacity catheter stage 650 by imparting a force F6 that has a lateral component relative to first catheter stage longitudinal axis at first catheter stage 605, for example, as shown as F6. This causes first catheter stage 605 to pivot away from high-capacity catheter stage 650 in direction D6 to a position as shown in FIG. 6b.

FIG. 6b is a side view of the two-stage suction catheter as shown in FIG. 6a after separation of the two stages by imparting force F6. After this separation, high-capacity catheter stage 650 is available for use in applying high capacity suction to rapidly clear liquids and solids as necessary.

FIG. 7a is a side view of an alternative attachment arrangement of a two-stage suction catheter 700 where the attachment arrangement 730 comprises a hinged connection 768. The respective lumens of first catheter stage 705 and high-capacity catheter stage 750 are coupled by compression and are held together by hinged connection 768. First catheter stage 705 may be separated from high-capacity catheter stage 750 by imparting a force F7 that has a lateral component relative to first catheter stage longitudinal axis at first catheter stage 705, for example, as shown as F7. This causes first catheter stage 705 to pivot away from high-capacity catheter stage 750 in direction D6 to a position as shown in FIG. 7b.

FIG. 7b is a side view of the two-stage suction catheter as shown in FIG. 7a after separation of the two stages by imparting a force by imparting force F7. After this separation, high-capacity catheter stage 750 is available for use in applying high capacity suction to rapidly clear liquids and solids as necessary.

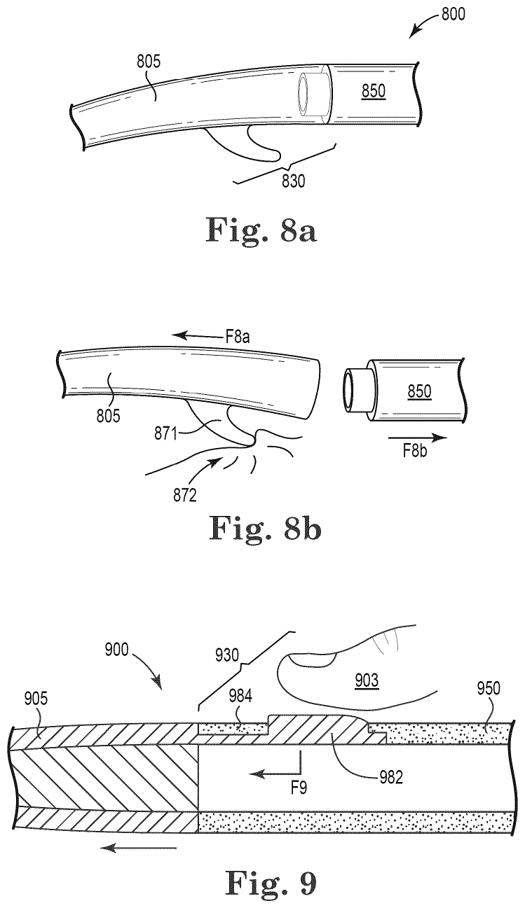

FIG. 8a is a side view of a two-stage suction catheter 800 where the attachment arrangement 830 comprises a sliding connection where a portion of high-capacity catheter stage 850 fits inside first catheter stage 805. First catheter stage 805 comprises a release-assist projection 871 that extends laterally from an external surface of the first catheter stage 805.

Separation of first catheter stage 805 from high-capacity catheter stage 850 is carried out by imparting a force F8a to first catheter stage 805, wherein force F8a has a longitudinal component parallel to the longitudinal axis of the first catheter stage in the direction away from the high-capacity catheter stage 850. The force may be applied by, for example, by catching the release-assist projection 871 on an available structure 872 separate from the two-stage suction catheter, such as an available table, support stand, bed frame or (as shown in the figure) the mattress of the gurney on which the patient is positioned. The practitioner pulls on the high-capacity catheter stage 850 in a direction away from first catheter stage 805, shown as force F8b. Release-assist projection 871 is shown in the shape of a hook, but may be in any shape or orientation having a lateral orientation relative to the external surface of the first catheter stage 805 that facilitates engagement with a structure separate from the two-stage suction catheter that imparts a separating force.

FIG. 8b is a side view of the two-stage suction catheter 800 as shown in FIG. 8a after separation of the two stages. After this separation, high-capacity catheter stage 850 is available for use in applying high capacity suction to rapidly clear liquids and solids as necessary.

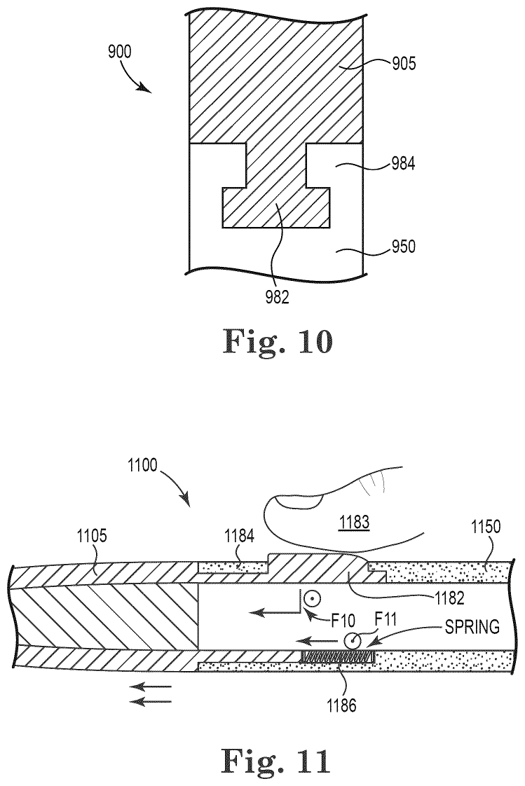

FIG. 9 is a side partial cross-section view of two-stage suction catheter 900, and FIG. 10 is a top view of two-stage suction catheter 900, where the attachment arrangement 930 comprises a thumb activated release. In this embodiment, flange portion 982 of first catheter stage 905 is depressed by the thumb 983 of a practitioner. Flange portion 982 is pushed into below retention wings 984 of high-capacity catheter stage 950. First catheter stage 905 is thereby released, and the practitioner then presses flange portion 982 forward and away from high-capacity catheter stage 950 in direction F9 to push first catheter stage 905 away from high-capacity catheter stage 950.

FIG. 11 is a side cross-section view of a two-stage suction catheter 1100, which is similar to the two-stage suction catheter as shown in FIGS. 9 and 10, except that two-stage suction catheter 1100 comprises an assisted thumb activated release.

In operation, flange portion 1182 of first catheter stage 1105 is depressed by the thumb 983 of a practitioner. Flange portion 982 is pushed into below retention wings 1184 of high-capacity catheter stage 1150 and acts as a release switch. First catheter stage 1105 is thereby released, and the practitioner then presses flange portion 1182 forward and away from high-capacity catheter stage 1150 in direction F10 to push first catheter stage 1105 away from high-capacity catheter stage 1150. Two-stage suction catheter 1100 is additionally provided with biasing member 1186 (shown in the form of a spring) configured to apply a force F11 to urge separation of first catheter stage 1105 from high-capacity catheter stage 1150 upon actuation of the release switch.

After this separation, high-capacity catheter stage 1150 is available for use in applying high capacity suction to rapidly clear liquids and solids as necessary.

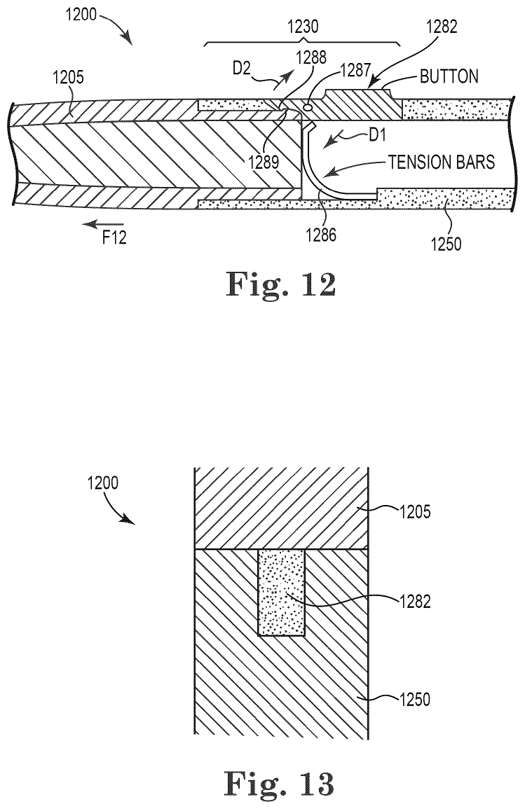

FIG. 12 is a side cross-section view of two-stage suction catheter 1200, and FIG. 13 is a top view of two-stage suction catheter 1200, where the attachment arrangement 1230 comprises a thumb activated release button 1282 connected to high-capacity catheter stage 1150 at pivot axis 1287. First catheter stage 1205 is releasably connected to high-capacity catheter stage 1250 by engagement of button latch 1288 with first catheter stage latch 1289. In this embodiment, when thumb activated release button 1282 is depressed by the thumb of a practitioner, the side of thumb activated release button 1282 adjacent to high-capacity catheter stage 1150 travels down in direction D1, and the side of thumb activated release button 1282 adjacent to first catheter stage 1205 travels up in direction D2 decoupling button latch 1288 from first catheter stage latch 1289 and releasing first catheter stage 1205 is thereby released.

Two-stage suction catheter 1200 is additionally provided with biasing member 1286 (shown in the form of a tension bar) configured to apply a force F12 to urge separation of first catheter stage 1205 from high-capacity catheter stage 1250 upon decoupling of button latch 1288 from first catheter stage latch 1289.

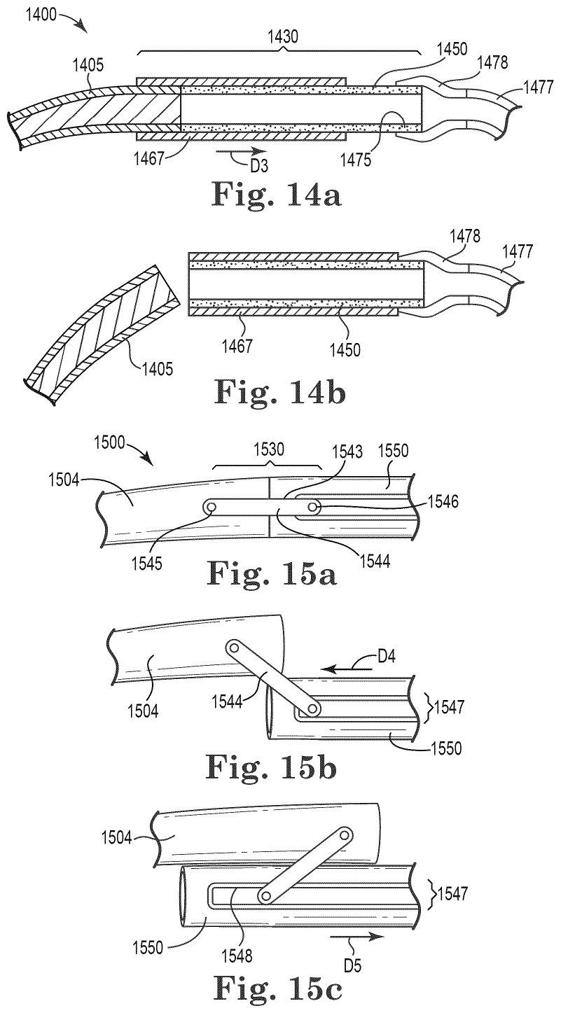

FIG. 14a is a side cross-section view of a two-stage suction catheter 1400 where first catheter stage 1405 is releasably connected to high-capacity catheter stage 1450 by the attachment arrangement 1430. High-capacity catheter stage 1450 is connected to high-capacity tubing 1477 by engaging tubing catheter connection end 1478 with High-capacity catheter stage connection end 1475.

Attachment arrangement 1430 comprises sliding barrel connection 1467 frictionally connects over first catheter stage 1405 and high-capacity catheter stage 1450 to provide temporary connection when in a first position, shown as a forward position in FIG. 14a. When the user recognizes the need for applying high capacity suction, sliding barrel connection 1467 may be slid to a second position to separate the first catheter stage from the high-capacity catheter stage. As shown, sliding barrel connection 1467 is slid back in direction D3 to release first catheter stage 1405 so that it simply falls away from high-capacity catheter stage 1450 as shown in FIG. 14b. In the event that sliding barrel connection 1467 is not completely withdrawn so that there is still some overlap of sliding barrel connection 1467 with first catheter stage 1405, the use can apply a lateral force such as by striking two-stage suction catheter 1400 on a surface such as mattress to complete separation of the two pieces.

FIG. 15a is a side view of an alternative attachment arrangement of a two-stage suction catheter 1500, where the respective lumens of first catheter stage 1505 and high-capacity catheter stage 1550 are coupled by compression and are held together by an attachment arrangement 1530. Attachment arrangement 1530 comprises track slide connector 1543 comprising bar 1544. Bar 1544 is attached to first catheter stage 1505 by first link 1545, and to high-capacity catheter stage 1550 by second link 1546. First link 1545 is attached to first catheter stage 1505 as a hinge to permit pivoting of track slide connector 1543 as shown in FIG. 15b. Second link 1546 is attached to track 1547 in high-capacity catheter stage 1550, and is configured to slide in the track in a direction D4 parallel to the longitudinal axis of the first catheter stage in the direction toward the distal end inlet of first catheter stage 1505, and also a direction D5 parallel to the longitudinal axis of the first catheter stage in the direction away from the distal end inlet of first catheter stage 1505. Projection 1548 is located in track 1547 to resist sliding movement of second link 1546 in track 1547, thereby maintaining compressive coupling of first catheter stage 1505 and high-capacity catheter stage 1550.

FIG. 15b shows the initial step of separation of first catheter stage 1505 from high-capacity catheter stage 1550, wherein a force is imparted having a longitudinal component parallel to the longitudinal axis of the first catheter stage in the direction away from the high-capacity catheter stage 1550, so that first catheter stage 1505 is caused to travel in a direction D4 to overcome the detent represented by projection 1548. As shown in FIG. 15c, first catheter stage 1505 is then spatially displaced from high-capacity catheter stage 1550 and withdrawn by traversal of second link 1546 down track 1547 in the direction D5. In an embodiment, first catheter stage 1505 is flipped so that the distal end inlet of first catheter stage 1505 is pointed in direction D5 before withdrawal by traversal of second link 1546 down track 1547.

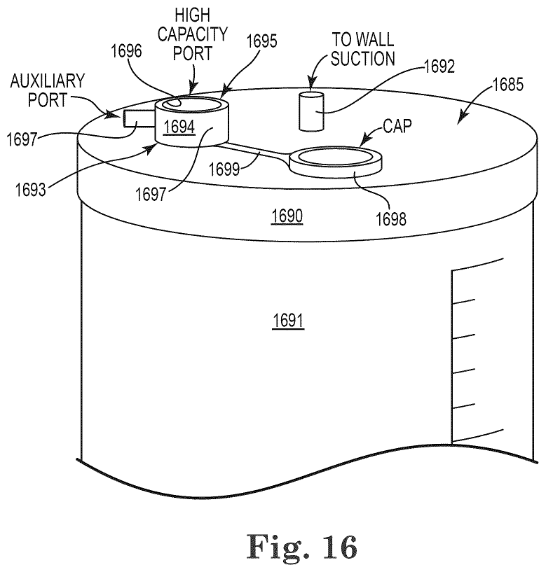

FIG. 16 is a perspective view of an embodiment of a suction cannister lid 1690 attached to suction cannister 1691. Suction cannister lid 1690 is provided with wall suction port 1692 for fluid communication of suction cannister 1691 to a vacuum supply (not shown).

Vacuum port array 1693 includes body portion 1694 formed as a hollow cylinder having an inner diameter 1696, the body portion 1694 extending from the top surface 1685 of cannister lid 1690. Body portion 1694 defines high capacity port 1695 providing fluid communication through cannister lid 1690. In an embodiment, high capacity port 1695 has an inner diameter of from about 14 mm to about 18 mm. In an embodiment, high capacity port 1695 has an inner diameter of from about 15 mm to about 17 mm.

At least one auxiliary port 1697 extends laterally from body portion 1694. The at least one auxiliary port 1697 may be used for alternative suction applications using tubing of different sizes in suction procedures where there is no need for the availability of high suction capacity. Thus, the present suction cannister lid 1690 is useful for multiple different applications, including high suction and low suction applications, without the need for the medical facility to stock a number of suction cannister lid products.

In order for the auxiliary ports to operate, high capacity port 1695 is closed by high capacity port cap 1698. High capacity port cap 1698 is optionally attached to cannister lid 1690 with cap tether 1699. In an embodiment, one or more auxiliary ports are provided having inner diameters of from about 5 mm to about 7 mm.

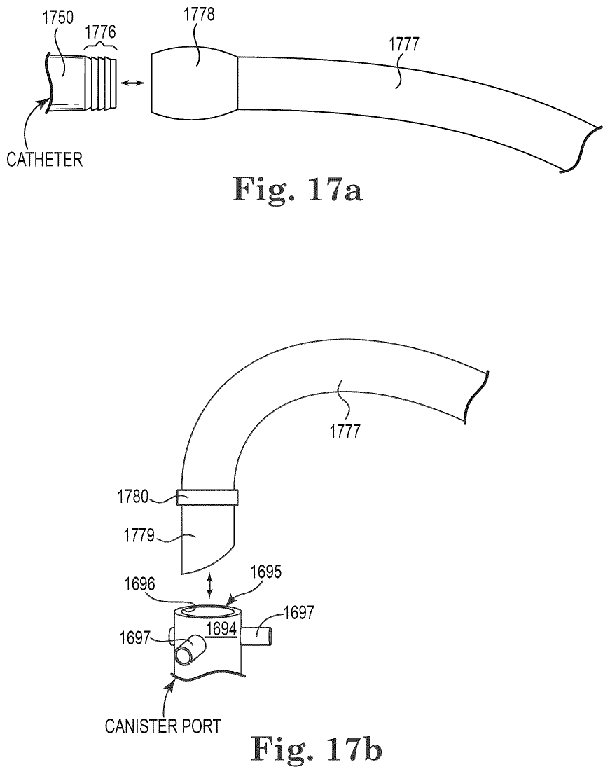

Similarly, for high capacity port 1695 to operate, all auxiliary ports must be closed. If an auxiliary port is open, flow of air and suctioned material through the high-capacity system it will decrease and can cause system failure. FIGS. 17a and 17b show a catheter tubing arrangement whereby auxiliary ports are closed automatically when high capacity port 1695 is in use.

As shown in FIG. 17a, high-capacity catheter stage 1750 comprises hose barbs 1776 for connecting high-capacity catheter stage 1750 to high capacity tubing 1777 via tubing catheter connection end 1778.

As shown in FIG. 17b, high capacity tubing 1777 is provided with tubing cannister connection end 1779 for connecting with high capacity port 1695. High capacity port 1695 has an inner diameter 1696 having a size relative to the outer diameter of tubing cannister connection end 1779 so that an airtight coupling seal is formed when tubing cannister connection end 1779 is inserted into high capacity port 1695. In an embodiment, high capacity tubing 1777 is provided with flange 1780 spaced from tubing cannister connection end 1779 to define an insertion depth of high capacity tubing 1777 into high capacity port 1695 and to assist in formation of an airtight coupling seal. Upon insertion of tubing cannister connection end 1779, auxiliary ports 1697 are occluded so that no air flows through auxiliary ports 1697.

In an embodiment, high capacity tubing 1777 has an inner diameter of from about 14 mm to about 18 mm. In an embodiment, capacity tubing 1777 has an inner diameter of from about 15 mm to about 17 mm. Because the wall suction has a maximum negative pressure of 200 mmHg, it is important to minimize the resistance of flow in the tubing to avoid collection of vomitus and debris in the tubing that can lead to system failure. In an embodiment, high capacity tubing 1777 is provided with a smooth, low-friction inner surface.

In an embodiment, suction cannister lid 1690 or suction cannister 1691 comprise additional functional features, such a drain port.

As used herein, the terms “about” or “approximately” mean within an acceptable range for the particular parameter specified as determined by one of ordinary skill in the art, which will depend in part on how the value is measured or determined, e.g., the limitations of the sample preparation and measurement system. Examples of such limitations include preparing the sample in a wet versus a dry environment, different instruments, variations in sample height, and differing requirements in signal-to-noise ratios.

All patents, patent applications (including provisional applications), and publications cited herein are incorporated by reference as if individually incorporated for all purposes. Unless otherwise indicated, all parts and percentages are by weight and all molecular weights are weight average molecular weights. The foregoing detailed description has been given for clarity of understanding only. No unnecessary limitations are to be understood therefrom. The invention is not limited to the exact details shown and described, for variations obvious to one skilled in the art will be included within the invention defined by the claims.

1. A two-stage suction catheter for rapidly clearing liquids and solids from the upper airway comprising:
a first catheter stage having a tapered lumen extending from a distal end inlet to an attachment end outlet, wherein the distal end inlet has an aperture size inner diameter of from about 5 to about 7 mm and the attachment end outlet has an aperture size inner diameter of from about 14 to about 18 mm, the first catheter stage having a longitudinal axis defined as a line passing through the center of the lumen, the first catheter stage comprising a first catheter stage airflow vent configured regulate effective negative pressure level at the distal end inlet when the two-stage suction catheter is connected to a vacuum supply;
a high-capacity catheter stage having a lumen extending from a distal connection inlet to a proximal end outlet, wherein the distal connection inlet has an aperture size inner dimension matching the aperture size inner dimension of the first catheter stage attachment end outlet, and the proximal end outlet has an aperture size inner dimension of from about 14 to about 18 mm, the high-capacity catheter stage comprising a high-capacity catheter stage ventilation aperture configured to regulate effective negative pressure level at the high-capacity catheter distal connection inlet when the first catheter stage is separated from the high-capacity catheter stage and the high-capacity catheter stage is connected to a vacuum supply; and
an attachment arrangement comprising a detent that releasably connects the attachment end outlet of the first catheter stage to the distal connection inlet of the high-capacity catheter stage, wherein the detent is configured to permit separation of the first catheter stage from the high-capacity catheter stage by a one-handed separation action.

2. The two-stage suction catheter of claim 1, wherein separation of the first catheter stage from the high-capacity catheter stage may be carried out by imparting a force to the first catheter stage that overcomes the detent, wherein the force has a lateral component relative to the longitudinal axis of the first catheter stage.

3. The two-stage suction catheter of claim 1, wherein separation of the first catheter stage from the high-capacity catheter stage may be carried out by imparting a force to the first catheter stage that overcomes the detent, wherein the force has a longitudinal component parallel to the longitudinal axis of the first catheter stage in the direction away from the high-capacity catheter stage.

4. The two-stage suction catheter of claim 1, wherein the attachment arrangement comprises a protuberant tab is provided on one stage of the two-stage suction catheter that is configured to engage with a corresponding recess provided on the other stage of the two-stage suction catheter as a detent to resist separation of the first catheter stage from the high-capacity catheter stage.

5. The two-stage suction catheter of claim 1, wherein the attachment arrangement comprises break-away connection struts configured to break upon application of force to separate the first catheter stage from the high-capacity catheter stage.

6. The two-stage suction catheter of claim 1, wherein the attachment arrangement comprises a connecting partial skirt component having a gap on one stage of the two-stage suction catheter to receive a cylindrical portion component on the other stage of the two-stage suction catheter to form a nested partial skirt coupling arrangement.

7. The two-stage suction catheter of claim 1, wherein the attachment arrangement comprises a hinged connection coupling the first catheter stage and the high-capacity catheter stage together in compression, such that the first catheter stage and the high-capacity catheter stage may be separated imparting a force having a lateral component relative to the first catheter stage longitudinal axis.

8. The two-stage suction catheter of claim 3, wherein the attachment arrangement comprises a sliding connection where a portion of one stage of the two-stage suction catheter fits inside a portion of the other stage of the two-stage suction catheter, and wherein the first catheter stage comprises a lateral projection relative to an external surface of the first catheter stage configured to engage with a structure separate from the two-stage suction catheter to impart the force having a longitudinal component in the one-handed separation action.

9. The two-stage suction catheter of claim 1, wherein the attachment arrangement comprises a release switch configured to disable operation of the detent and permit separation of the first catheter stage from the high-capacity catheter stage.

10. The two-stage suction catheter of claim 10, wherein the attachment arrangement comprises a biasing member configured to apply a force to urge separation of the first catheter stage from the high-capacity catheter stage upon actuation of the release switch.

11. The two-stage suction catheter of claim 9, wherein the release switch comprises a thumb activated release button.

12. The two-stage suction catheter of claim 1, wherein the attachment arrangement comprises a sliding barrel connection over the first catheter stage and the high-capacity catheter stage to provide temporary frictional connection when in a first position, wherein the sliding barrel connection may be slid to a second position to separate the first catheter stage from the high-capacity catheter stage.

13. The two-stage suction catheter of claim 1, wherein the attachment arrangement comprises a track slide connector coupling the first catheter stage and the high-capacity catheter stage together in compression, comprising a bar attached to first catheter stage by a first link as a hinge to permit pivoting of track slide connector, and to high-capacity catheter stage by a second link configured to slide in the track in a direction parallel to the longitudinal axis of the first catheter stage in the direction toward the distal end inlet of first catheter stage, and also a direction parallel to the longitudinal axis of the first catheter stage in the direction away from the distal end inlet of first catheter stage, wherein a projection is provided in the track to resist sliding movement of second link in track, thereby maintaining compressive coupling of first catheter stage and high-capacity catheter stage.

14. A method of rapidly clearing liquids and solids from the upper airway comprising:
applying suction to a patient in need thereof with the two-stage suction catheter of claim 1 using the first catheter stage;
detaching the first catheter stage from the high-capacity catheter stage in a one-handed separation action; and
applying suction to a patient in need thereof with the high-capacity catheter stage of the two-stage suction catheter.

15. A system for rapidly clearing liquids and solids from the upper airway comprising:
the two-stage suction catheter of claim 1;
a suction cannister system comprising a suction cannister and suction cannister lid, the suction cannister lid comprising a high capacity port; and
high-capacity tubing configured to connect the proximal end outlet of the high-capacity catheter stage of the two-stage suction catheter to the high capacity port to form an airtight coupling seal from the two-stage suction catheter to the high capacity port.

16. The system of claim 15, wherein the suction cannister lid comprises
a wall suction port configured for attachment to a vacuum supply;
a vacuum port array comprising
a body portion formed as a hollow cylinder extending from a top surface of the cannister lid defining a high capacity port providing fluid communication through the cannister lid, and
an auxiliary port extending laterally from the body portion,
the high capacity port being configured to receive the high-capacity tubing inserted into the high capacity port to form an airtight coupling seal permitting flow of air and suctioned material through the high capacity port and simultaneously occluding the auxiliary port to prevent air flow through the auxiliary port; and
a removable cap configured to seal the high capacity port while leaving the auxiliary port open permitting flow of air and suctioned material through the auxiliary port.

17. A suction cannister lid comprising
a wall suction port configured for attachment to a vacuum supply;
a vacuum port array comprising
a body portion formed as a hollow cylinder extending from a top surface of the cannister lid defining a high capacity port providing fluid communication through the cannister lid, and
an auxiliary port extending laterally from the body portion,
the high capacity port being configured to receive high-capacity tubing inserted into the high capacity port to form an airtight coupling seal permitting flow of air and suctioned material through the high capacity port and simultaneously occluding the auxiliary port to prevent air flow through the auxiliary port; and
a removable cap configured to seal the high capacity port while leaving the auxiliary port open permitting flow of air and suctioned material through the auxiliary port.

18. The two-stage suction catheter of claim 10, wherein the release switch comprises a thumb activated release button
